(19) United States
(12) Patent Application Publication
(10) Pub. No.: US 2017/0065438 A1
(43) Pub. Date: Mar. 9, 2017

(54) Adjustable tibial trial

(71) Applicant: Brian G. Burnikel, Greer, SC (US)

(72) Inventor: Brian G. Burnikel, Greer, SC (US)

(21) Appl. No.: 14/756,476

(22) Filed: Sep. 8, 2015

(51) Int. Cl. A61F 2/46

(52) U.S. Cl. CPC A61F 2/4684; A61F 2/461; A61F 2/4657; A61F 2002/4628; A61F 2002/4661

(57) ABSTRACT

An adjustable, temporary device that exerts spacing force through a pair of identical lattices to increase the distance between the distal femur and proximal tibia. Pairs of lattices are connected to a femoral and a tibial base plate by connections to lift anchors. A pair of connector rods joins the lattices; the drive rod that includes a travel block functionally joins the connector rods. The threaded length of drive rod engages the threaded chase of the travel block and the smooth second end engages the smooth chase of the static block. The chases are aligned at a right angle to the anchor rods. Rotating the anchor rod moves the travel block so that a forces is exerted on the lattices. Rotating the drive rod increases the length of the lattices and the distance between the femur and tibia which is expressed as increased tension on the ligaments and soft tissue of the knee. The device may include a measuring tape housing with a flexible tape. When the surgeon determines by manipulation of the knee joint that the optimum tension has been reached, the distance is measured and the optimum thickness of the permanent spacer is established.

### PRIORITY

This Application claims priority of U.S. Provisional Application No. 62/070,983 filed Sep. 10, 2014 and which Provisional Application is hereby incorporated in its entirety, by reference.

### INTRODUCTION

Field of the Invention

The invention is related to orthopedic surgery. More specifically it is related to knee replacement surgery, and specifically it is related to a device that will allow the surgeon to determine accurately, during surgery the optimum thickness of the spacer that is an element of knee replacement surgery.

Background of the Invention

A variety of conditions may lead to knee replacement surgery; they share a common, undesirable characteristic—pain that ultimately interferes with daily activities and recreational activities. Injuries and infections can lead to knee replacement surgery, but the most common cause is arthritis in one of its varied forms- the most common form, osteoarthritis, rheumatoid arthritis, and traumatic arthritis.

Knee replacement surgery has become the most common form of joint replacement surgery in the United States (and more than likely, world wide). In the United States in excess of 720,000 procedures were preformed in the past several years with a reported success rate of 80 to 90 percent. Success is not measured as return of perfect function, but most commonly as marked relief from bone-on-bone induced pain, improved joint flexibility, “near normal” function, considering the age, weight, and general health of the patient.

Understanding the fundamental anatomy of the knee and major steps in replacement surgery provides a necessary foundation for understanding the structure and function of the adjustable tibial trial disclosed and claimed in the specification and appended claims hereof. As noted below under Brief Description of then Drawings, FIGS. 1-5 present basic background material necessary to understanding the invention and FIGS. 6-10 describe and explain the how to make and use the invention in detail.

The knee 101 is among the largest and most complex joints in the human body, FIG. 1. The knee connects the femur (thigh bone) 102 to the tibia (shin bone) 103. The knee includes a third bone, the patella (knee cap) 104, a relative small oval (nodule) of bone that lies within a tendon and slides along the surface of another bone, the femur 103 as the knee joint is bent or flexed. These three bones provide the mechanical frame or structure of the knee joint 101. The fibula is a smaller bone extending from the top (proximal) end of the femur 102 to the ankle (talus). Commonly, the fibula is not considered to be part of the knee 101.

Mechanically, the knee 101 is described as a hinge joint or a modified hinge joint, by analogy, something like the common door hinge. Of course, the knee can do more than merely swing back and forth. An amazing network of muscles, ligaments, and tendons permits the knee to rotate in conjunction with flexing and/or extending the knee. In addition, ligaments, tendons, and associated muscles provide strength and stability to the joint. In terms of major support of the knee, as illustrated in FIG. 2, the anterior cruciate ligament and the posterior cruciate ligament, 106 and 107 respectively, keep the femur 102 from sliding backwards on the tibia 103 and keep the femur 102 from sliding forward on the tibia 103. The medial and lateral collateral ligaments, 108 and 109, respectively, effectively prevent sliding laterally. The thigh muscle(s) (not illustrated) play a key role in the mechanical function of the knee 101.

Normal activity potentially exposes the distal surfaces 110 of the femur 102 and the proximal surface 111 of the tibia 103 to mechanical (jolting) wear. The medial meniscus 112 and the lateral meniscus 113 are positioned to function as shock absorbers between the distal end 110 of the tibia and the proximal end 111 of the femur 102. These meniscuses are crescent, or block “C” shaped cartilage bodies formed from fibrous cartilage. Essentially all boney surfaces that contact another boney surface in the knee (as well as in other joints) are coated with a layer of hyaline cartilage 114 that produces a smooth surface on which opposing boney surfaces move smoothly one against the other.

Neither type of cartilage is indestructible. The medial 112 and lateral meniscus 113 are subject to tear, and the fibrous cartilage of the meniscuses and the hyaline cartilage that protects various surfaces of the knee are all subject to arthritic degeneration and resulting damage to the knee joint surfaces. Injuries, heavy lifting, and repeated, high impact or constantly twisting activities involving the knee may cause damage or accelerate the development of arthritis. The reasons for surgery are well known: pain relief, improved joint stability, improved joint alignment and bone deformity, and enhanced general quality of life.

The exact surgical procedure in a knee replacement operation varies among surgeons and with specific conditions of the individual patient; however, the basic steps are quite consistent. Understanding the basic steps is important to understanding and appreciating the invention as represented in the appended claims.

The minimally invasive knee replacement surgical procedure is base on the premise that the incision down the front of the knee is held to a minimum length; however relative length may vary among patients. The surgery seeks to minimize the amount of bone removed, but recognizes the need to remove all damaged tissue that might affect the outcome of the surgery. In addition, hospitalization is minimized and recovery time significantly reduced.

FIG. 3 illustrates the knee 101 of FIG. 2 in which, in FIG. 3, the medial meniscus 112 and the hyaline cartilage surface 114 are stippled to indicate severe arthritic damage to the cartilage as well to the surrounding bone. In both FIG. 2 and in FIG. 3, the distal surface of the femur 110 and the opposing proximal surface of the tibia 111 are shown as being physically separated, points “A” and “B,” 115A and 115B, respectively. This separation is to simplify the illustrations.

In a healthy, non-arthritic joint, such as FIG. 2, the distal surface of the femur 110 would be cushioned by the shock absorbing capacity of the fibrous cartilage and the lateral meniscus 113 on the proximal surface of the tibia 103. As suggested in FIG. 3, with “normal spacing” and the damage to the cartilage and bones as a result of advanced arthritis , the distal surface of the femur 110 and proximal surface of the tibia 111 as indicated by “A” and “B” 115A and 115B, respectively, would be bone-to-bone contact resulting in severe pain, markedly when going up or down stairs, twisting, or carrying weight or other high impact activities. This constant discomfort and restrictions on activities from the bone-on-bone contact is a major reason for knee replacement surgery.

A knee replacement prosthesis comprises three basic components: the femoral component (related to the thigh bone); the tibial component (related to the tibia); the tibial component usually comprises two components: a flat, metal or alloy plate that is attached to the bone and a spacer usually made from a highly durable plastic that provides a smooth surface over which the fibular element moves; and the patellar component (related to the knee cap). Unfortunately, in selecting an appropriate prosthetic implant (knee joint), it is not a simple matter that one size fits everyone. In response to the explosive growth of the number of knee replacement surgeries performed since the early 1970's and the recognition of the wide scope of variation among individuals in knee size and shape, prosthetic research and development and manufacturing and supply companies world wide have implant elements such that nearly all (about 98%) of the implants needed can be satisfied by one or more existing size or shape devices.

Determining the appropriate size for the femoral, tibial, and patellar components starts before surgery with measurements made from various x-ray views of the joint and actual leg measurements. Current technology includes computer analysis of x-ray using a fixed size reference in the x-ray to measure bone (joint) surface areas as well as circumferences of joints that are not regularly round, thereby reducing the need for trial and error fitting of some components of the prosthetic device (joint).

The initial measurements made from pre-operative x-rays include full consideration of the amount of diseased, injured, or weakened bone must be removed from the femur, tibia, and patella, but absolute amounts cannot be confirmed before surgery. Final fitting of the prosthetic device is a critical part of the surgical procedure. Trial components are inserted as part of the process to confirm appropriate fit of specific sizes of the components, and final adjustments are made to the overhang of the bone over the prosthetic component (element too small) or extension of the element over the bone (underhung, component too large), either of which conditions may cause continued, post-operative pain. Skilled surgeons can adjust positioning of the component and make slight additional tissue removals to ensure an optimum fit. The final step in fitting the prosthetic device is determining the optimum thickness of the spacer part of the fibular component.

FIG. 2 illustrates a healthy knee joint. Spaces “A” and “B” 115A and 115B, respectively are shown for convenience and reference. Normally, the distal surface of the femur 110 is in direct contact with the medial meniscus and lateral meniscus 112 and 113, respectively, and the open areas A and B (115A and 115B) are not seen (do not exist as open areas.)

FIG. 3 illustrates the same knee joint as in FIG. 2 except the joint in FIG. 3 displays extensive arthritic damage to the distal surface of the femur 114 and proximal surface of the tibia and to the medial meniscus and lateral meniscus 114A, 112A, and 113A, respectively. These areas are the area from which the affected (arthritis) bone is shaved and the femur and tibia 116 and 117, respectively, surgically shaped to allow precise fitting of the femural and tibial components of the prosthesis, as illustrated in FIG. 5.

Two types of knee replacement surgeries should be considered: initial joint replacement surgery, or arthroplasty and replacement of a damaged prosthetic joint, revision surgery. In the former case, commonly fitting the prosthetic device requires less removal of bone, and the size (thickness) of the required spacer can be quite accurately estimated before surgery; whereas, revision procedures bone (tibia) removal may be more extensive as a result of injury or infection (or similar wear and tear) on older prosthetic devices. In addition to secure properly a tibial component, removal of more bone may be required to ensure adequate bone strength to hold the component. Regardless, final determination of the optimum thickness of the spacer must be accomplished as a part of the surgical procedure. Until the femur and tibia are shaped and the corresponding components implanted, the thickness of the spacer cannot be finitely determined.

The lateral stability of the prosthetic knee depends largely on the lateral, collateral ligaments 109 and medial collateral ligaments 108 (FIG. 2). The medial collateral ligament 108 is on the inside of the knee, extending from the femur 102 to the tibia 103, and the lateral collateral ligament is on the outside of the knee, extending from the femur to a point near the head of the fibula 105. In the majority of the surgeries, the anterior and posterior cruciate ligaments are removed and the prosthesis is designed to compensate for this. The patella and its supporting ligaments are rarely an issue in with respect to the thickness of the spacer; therefore, the are not considered further in the following discussion.

The optimum thickness of the spacer is simply defined or described: that thickness that separates the femoral component from the tibial component so that the tension on the medial collateral ligament 108 and lateral collateral ligament 109 optimum for medial and lateral support of the knee joint, without restriction of normal knee movement. With current technology, the surgeon, based on experience, the type of prosthesis implanted, and a “best estimate” inserts a “trial” spacer of known dimension (thickness), for example, 8 mm and manually tests the knee for flexibility and the ligaments for “tension.” If the joint is too loose, the trial spacer is removed, and a second, thicker, trial spacer is inserted, for example 12 mm, and the joint again tested. If it still is too lose, a third, thicker spacer, 16 mm, is tried. If, however, the joint is too tight, the 12 mm spacer is removed and a 10 mm spacer is tested. Optimum, proper, fit may require multiple trial reduction before the best thickness is established and the permanent spacer inserted and secured. As one skilled in the art understands, dimensions of the spacer other than thickness are important to ensure proper alignment of the femur and tibia components.

### PRIOR ART

The history and prior art of the knee spacer device is part of the history of joint replacement surgery and the parallel evolution of prosthetic devices and methods. The recognizes pioneer of knee replacement surgery is Leslie G. P. Shiers whose pioneering work was reported in 1954. Success with his hip replacement surgery in the 1960s sparked continuing attempts to design knee replacements, with innovations addressing problems of excess wear and loosening and the need for replacements that did not restrict the range of motion of the knee following revision surgery. Determining the optimum thickness for the spacer between the reshaped femur and tibia with or without elements of prosthetic implants in place is recognized as a continuing, significant element of successful knee replacement surgery.

U.S. Pat. No. 4,052,753 issued Oct. 11, 1977 to Dedo and titled, “Knee Spacer and Method of Reforming Sliding Body Surfaces,” discloses a temporary knee spacer with an elongated, flexible member manufactured from biologically, relatively inert material. The member is placed in the space normally occupied by the suprapatella pouch of the patient's knee. The removable device prevents adhesions between tendons and the femur following surgery. In addition, body tissues do not grow on the surface of the member, and the member causes the body to form sliding surfaces adjacent to the member surfaces during the healing process. In addition, the member is easy to remove from the joint after the sliding surfaces have been formed and the member facilitates the return to normal function of the joint.

U.S. Pat. No. 7,578,821 issued to Fisher, et al. On Aug. 25, 2009 and titled, “Dynamic Knee Balancer with Pressure Sensing describes a device for knee surgery that comprises an adjustable femoral portion, a tibial portion, and one or more sensors coupled with femoral and tibial portions that sense pressure exerted on the femoral and tibial portions on one another. The femoral portion is adapted to removably coupling with the distal end of the femur such that tension in soft tissue can be adjusted and has a positioning feature to move relative to the distal end of the femur, thereby helping to position a femoral prosthetic element on the distal end of the femur. Sensors are also adapted to measure pressure at the medial and lateral sides of the knee. Sensor data are presented by a visual display, and the femoral adjustment is used in balancing flexion and extension knee pressure.

The scope and role of the spacer element is clearly seen in U.S. Pat. No. 7,273,500, “Instruments and Methods for Use in Performing Knee Surgery,” issued Sep. 25, 2007 to Williamson. The disclosed invention utilizes a spacer element to guide horizontal femoral or tibial cuts. The invention comprises a system and method of ultimate alignment of the leg prior to making any cuts. The system comprises a spacer, a cutting guide, and a template. The spacer is inserted between the distal femur and proximal tibia to rotate the tibia into the desired alignment with the femur prior to making cuts; thus, when the prosthetic knee is implanted, the leg will be properly aligned.

Tarabichi in U.S. Pat. No. 7,455,647 issued Nov. 11, 2005 and titled, “Dynamic Spacer for Total Knee Arthroplasty.” describe and claim a spacer adapted to measure flexon-extension gap during total knee arthroplasty Two tissue engaging members are positioned, one at the distal end of the femur and the other at the proximal end of the tibia. A tension is disposed between the two members for applying tangible force between the members. The members are parallel, absent any compressible load. Measuring the flexion/extension gaps and angular deviation in flexion indicates the appropriateness of the established femoral rotation. A tensioning device is sandwiched between the first and second (femoral and tibial) members. The tensioning means may be coil springs, tensioning springs, and pneumatic devices, including air springs and pneumatic cylinders.

U.S. Pat. No. 7,470,288 issued to Dietz et al. On Dec. 30, 2008 and titled, “Telemetric Tibial Tray,” describes and claims a telemetric tibial tray comprising a plurality of cylindrical transducer cavities with circular load diaphragms. An upper plate is attached to the lower plate through support posts projecting from the load diaphragms. The support posts are circular in cross-section and about 5 mm in diameter. The lower plate also defines a wiring channel between the transducer cavity and the central cavities, housing the circuit board for the telemetric board. Each transducer cavity comprises a radial array of strain gauges with four pairs of radially aligned strain gauges. Each pair of strain gauges includes an inner gauge positioned at the point of maximum positive micro-strain across the diaphragm when loaded and a outer gauge positioned at the point of maximum negative micro-strain to increase the differential strain measured by the gauges and to increase the sensitivity of the tibial tray.

Recently, U.S. Pat. No. 8,656,790 issued to Amirouche on Feb. 25, 2014 and titled, “Device and Method of Spacer and Trial Design During Joint Arthroplasty,” discloses and claims a spacer block used to gather data to be be used in balancing joint arthroplasty or repair and in selection of trial inserts. The spacer block includes a first and a second body piece with a plurality of sensors positioned between the first and second body pieces when the device is assembled. A chim is removably mounted to a top surface of the second body piece; the chim is associated with the plurality of sensors and positioned in relation to the plurality of sensors such that a force exerted on the chim by a weight bearing surface is detected by the sensor.

### OBJECTIVES AND GOALS AND SUMMARY OF THE INVENTION

### Objective and Goals

A first objective of the invention is an adjustable, tibial trial device that is designed and adapted to allow the surgeon to increase (or within limits) to decrease the gap or space between the femur and tibia continuously, thereby allowing the surgeon to evaluate and compare the different spacings to identify the optimum spacing for function of the prosthetic knee; frequently, tension on supporting tendons is a major factor in making the determination of optimum spacing and thus on the size of the permanent spacer to ensure joint flexibility and alignment.

A second objective of the invention is a device that allows the surgeon to increase or decrease gap space to rapidly and repeatedly evaluate different potential gap spaces and the thickness of the permanent spacer to be inserted in the gap.

A third objective of the invention is a temporary, adjustable device that can change the gap space in small increments, either increasing or decreasing it to determine the optimum spacer thickness to be inserted.

A fourth objective of the invention is a device allows the surgeon to evaluate the effects of relative small changes in gap dimensions on relative tension on knee tendons.

The goal of the invention is a device that can be used before or after the permanent tibial and femoral elements of the prosthetic implant have been positioned, or prior to this stage as may be advisable or preferred by the surgeon, and only a measuring reference point need be changed to accommodate this modification.

### Brief Summary of the Invention

The stated objectives and goal of the invention and other implied objectives and goals are satisfied by a device that is adapted to increase or decrease the space between the distal femur and the proximal tibia thereby increasing or decreasing the tension on the ligaments and muscles that stabilize and support the knee joint; the device comprises a lower and an upper base plate and pairs of lower and upper anchor connector bracket, and a spacing mechanism; the spacing mechanism comprises a pair of extendable/retractable lattices with the top of each lattice attached to the upper anchor connectors and the bottom of each lattice attached to the lower anchor connectors; the lattices are joined by a pair of connecting rods on a pivot point on each lattice such that the members of the pair of connecting rods are parallel and in the common horizontal plane, the spacing mechanism further comprises a screw gear system, and the screw gear system comprises a drive rod with a first end and a second end in which about three-fourths of the length of the drive rod is threaded from the first end and the remaining one-fourth of the length is smooth; the screw gear system includes a travel block which is physically a part of the second drive connector rod and which includes a threaded chase bored at an horizontal, right angle to the length of the second connector rod, and which is functionally engaged by the first end of the drive rod; rotating the drive rod in a first direction causes the travel block to move towards the first connector rod that functions as a static block; the first connector rod move complimentary to the second connector rod; second end of the second drive rod is modified and adapted to allow manual rotation with a special tool; reversing the rotation to the second, opposite direction forces the pair of connector rods to move apart; the arrangement of the arms of the two trellises and arrangement of pivot points including the connections at the top and bottom of each trellis; movement of the members of the pair of connector rods inward is converted to increase in height (length) between the distal femur and proximal tibia; the distance can be measured by a flexible ruler folded into the ruler housing.

### BRIEF DESCRIPTION OF THE DRAWINGS

FIGS. 1-5 illustrate the basic structure of the knee joint and shaping of the femur and tibia to implant a prosthetic device. FIGS. 6-10 illustrate the adjustable tibial trial and its temporary placement in the knee joint.

FIG. 1 is a front (ventral) view of the bones of the knee joint.

FIG. 2 is a front (ventral) view of the knee joint including ligaments and cartilage.

FIG. 3 is a the same knee as FIG. 2 but showing the distribution and extensive damage from arthritis.

FIG. 4 illustrates with a front (ventral) view the surgical shaping of the femur and tibia in preparation for implanting an artificial knee joint.

FIG. 5 is a front view of the knee showing the femoral elements of the prosthesis in place.

FIG. 6 shows the knee with the adjustable tibial trial in place and partially extend disclosing the structure and major and elements of the device.

FIG. 7 illustrates a knee with the upper and lower base plates of the device in position.

FIG. 8 illustrates the lattice structure of the device with the gears connected to the anchor connector bracket.

FIG. 9A illustrates shows the adjustable tibial trial in a configuration in which gears illustrated in FIG. 8 are not used.

FIG. 9B illustrates details of the full lifting system that is used with the configuration illustrated in FIG. 9A.

FIG. 9C is a top view of the adjustable tibial trial mechanism nearly fully closed, with details of lifting device shown.

FIG. 10 illustrates one example of a tape housing structure, in this case the housing is shown as part of the upper base plate.

### ADJUSTABLE TIBIAL TRIAL

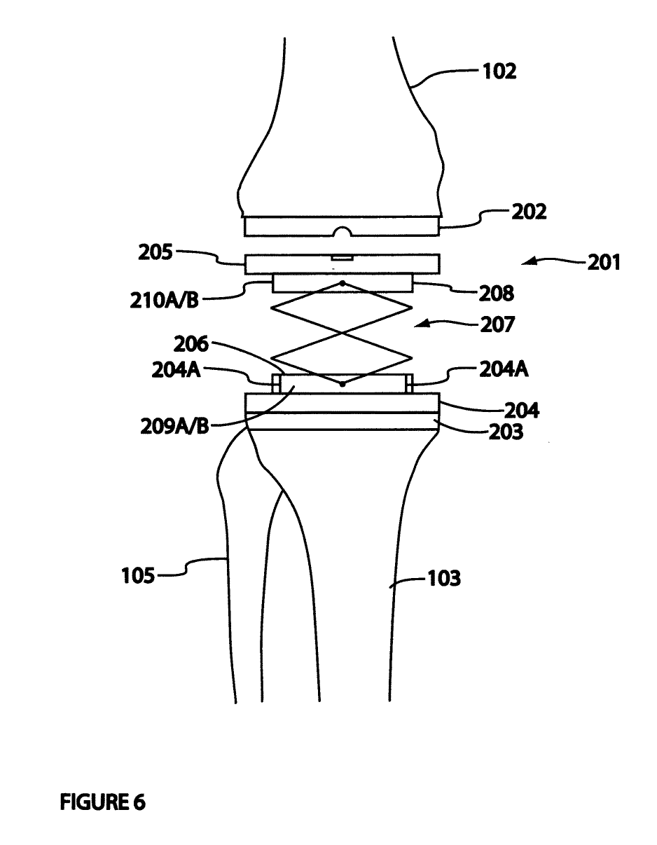

The thickness of the spacer is a direct function of the space (gap) or distance between the femur and tibia. This distance is frequently “measured” by the surgeon by manually testing the tension on the ligaments and soft tissue that stabilize and support the knee joint. The spacing device 201 provides a measurement of the optimum thickness of the space part of the tibia component as indicated by the surgeon's manual testing of tension and the change in tension associated directly with the space between the tibia and femur. The spacing device 201 comprises five major components, each of which may comprise more than one part. As illustrated in FIG. 6, the spacing device 201 comprises a lower base plate 204, an upper base plate 205, a lower spacing anchor 206, a spacing mechanism 207, and an upper spacing anchor 208.

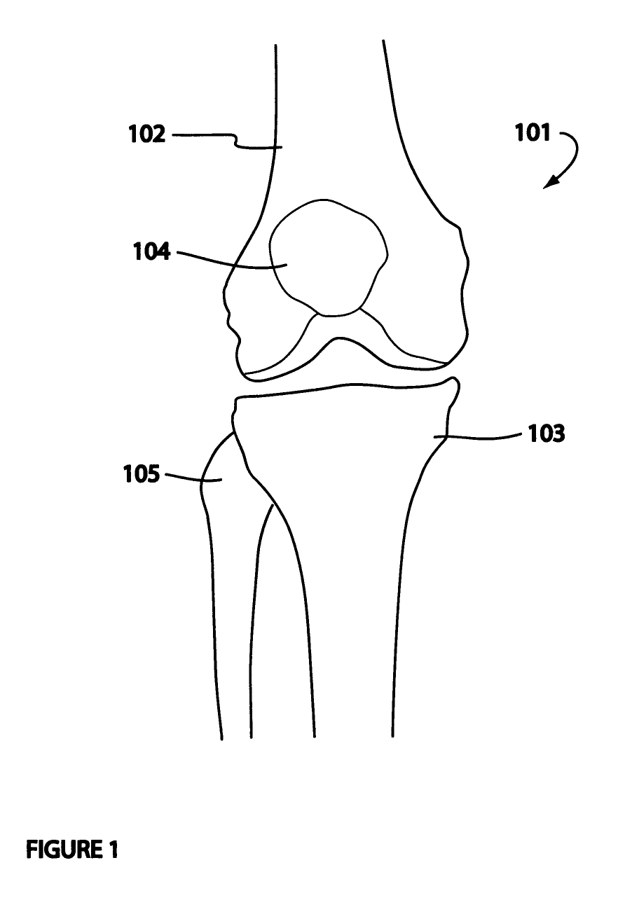

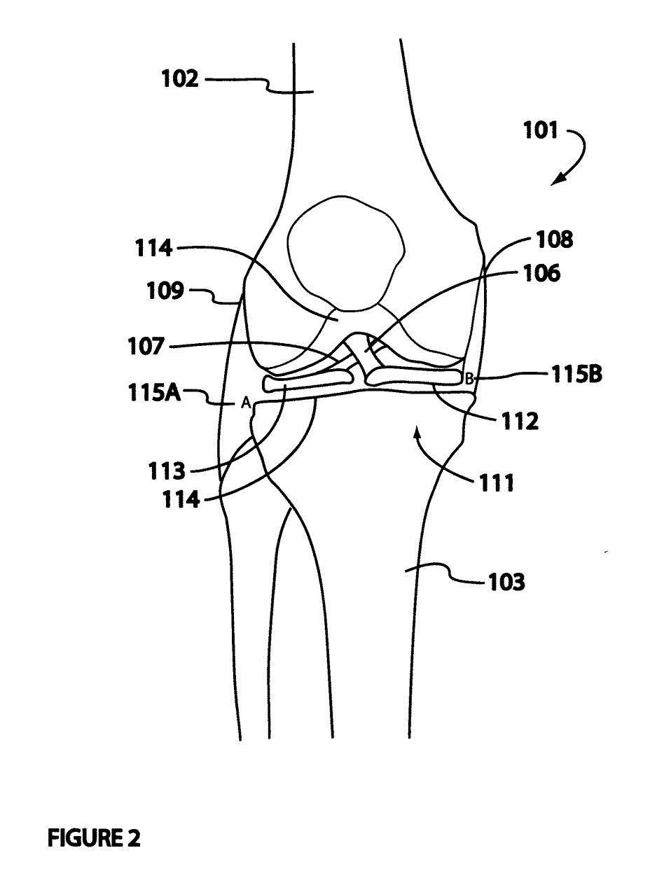

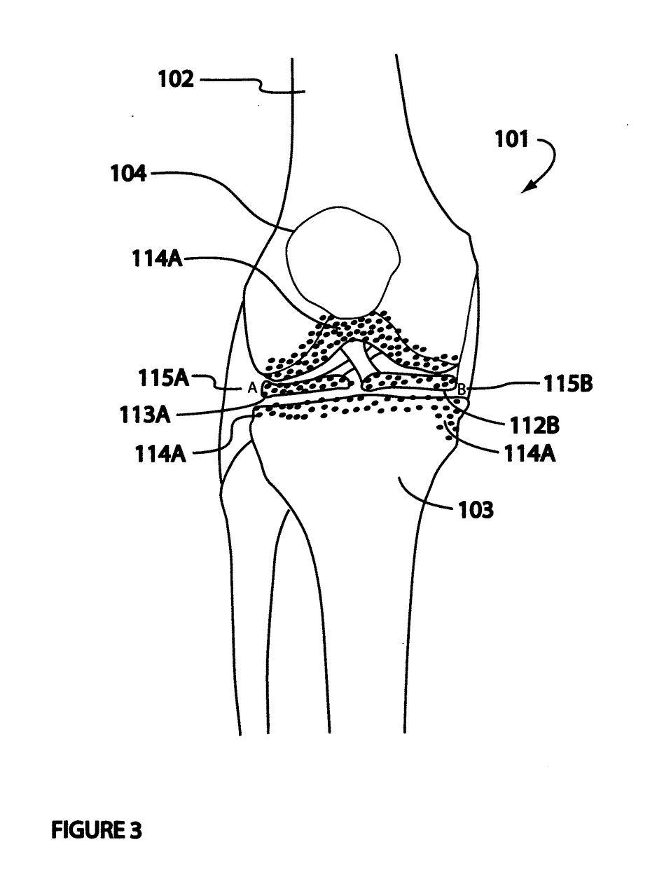

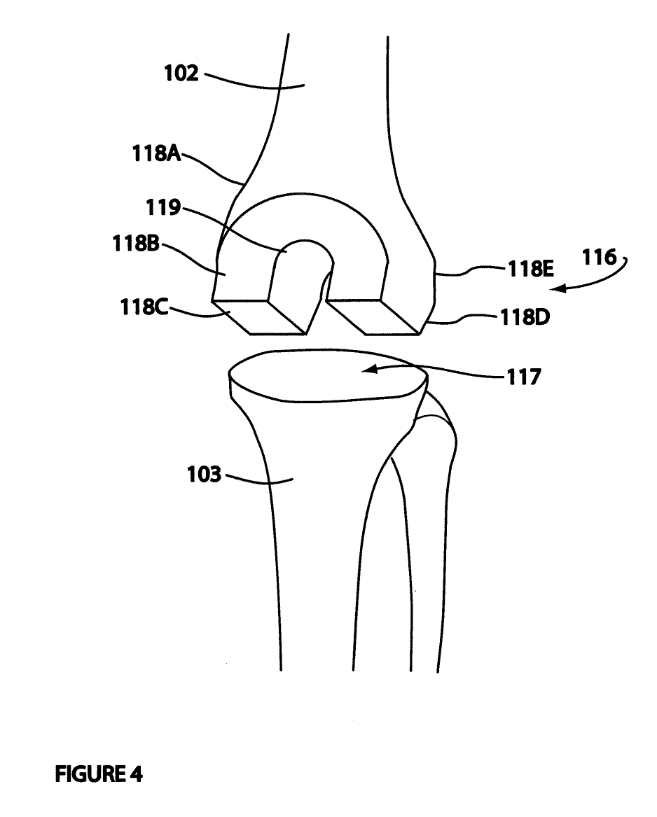

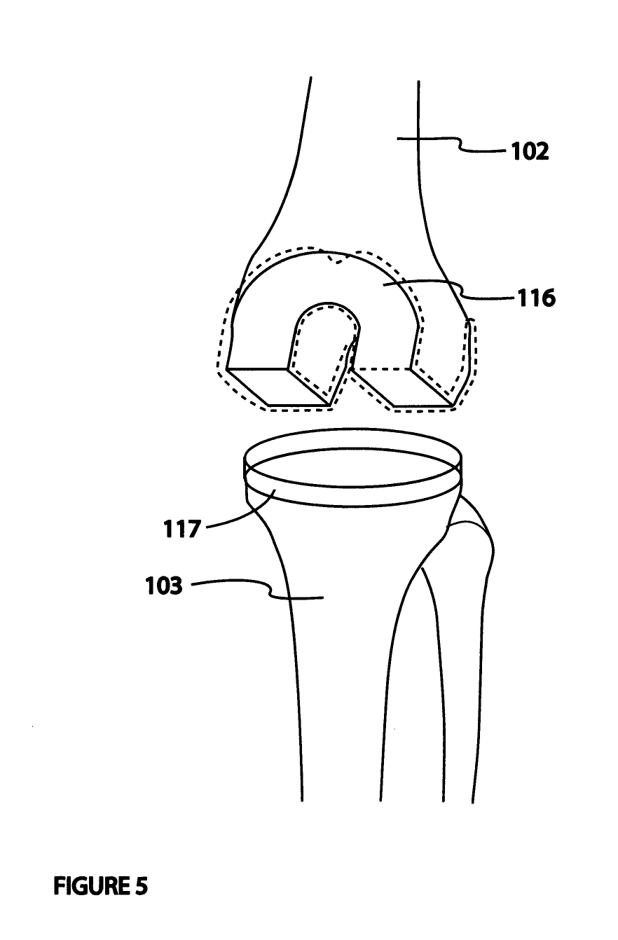

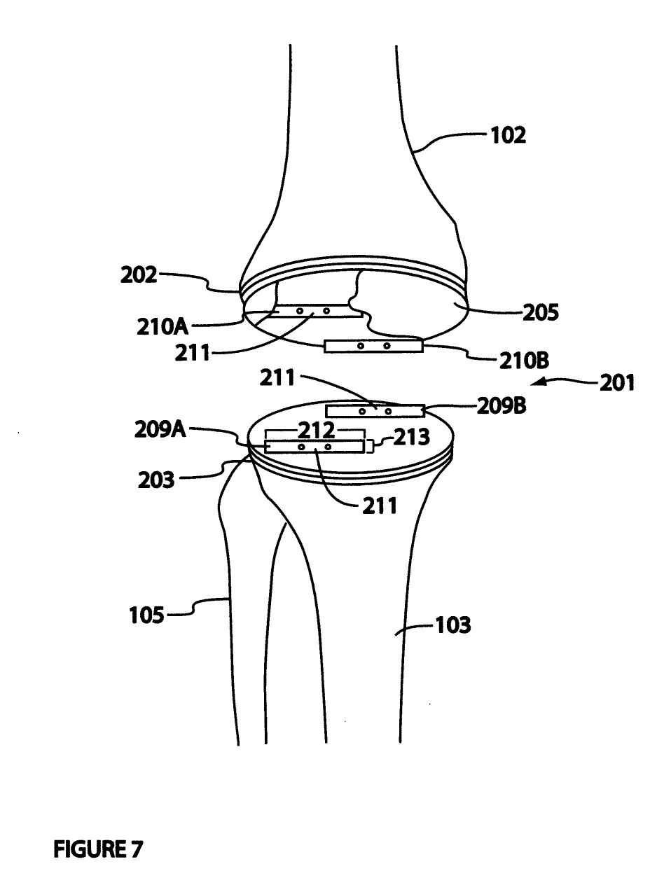

As illustrated in FIG. 6 and FIG. 7, both the permanent femoral component 202 and the permanent tibial component 203, are in place with the with the upper base plate 205 interfacing 205A with the femoral component 202. The lower base plate 204 may be mechanically connected to tibial component 203 by short bolts or screws 204A. The lower base plate 204 and upper base plate 205 may be connected directly and temporarily to the femur 102 and tibia 103 (FIG. 1). In this case, generally, the permanent femoral component 202 and tibial component 203 are not connected to the femur 102 and femur 103 until measurements and spacer considerations involving the Adjustable Tibial Trial 201 have been completed. The thickness of the permanent of the knee prosthesis parts must be included in final determination of the thickness of the spacers with thickness of all temporary parts deleted or accounted for in final spacing determinations. As one skilled in the art understands and appreciates, this option does not change or expand the purpose of the invention or the scope of the appended claims.

The lower spacing anchor 206 comprises an identical pair of anchor connector brackets 209A and 209B. Similarly, the upper spacing anchor comprises a second, identical pair of anchor connector brackets, 210A and 210B. FIG. 6 is a side view of the knee joint and shows only a single member of each pair of anchor connector brackets 209A/B and 210A/B. FIG. 7 provides a more detailed view of members of the pair of anchor connector brackets 209A and 209B positioned on and securely connected to the lower base plate 204 and the members of the pair of upper anchor connector brackets 210A and 210B.

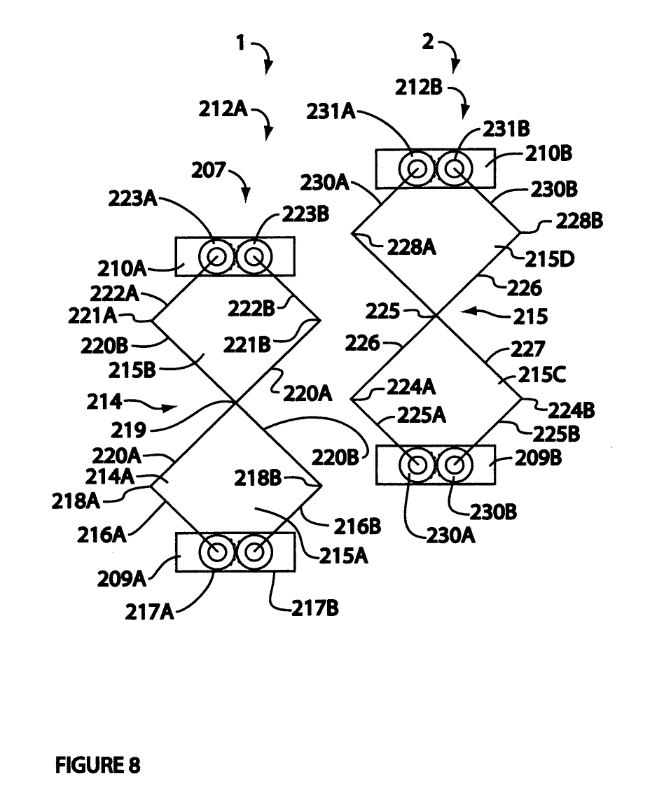

The spacing mechanism comprises a pair of extendable/retractable lattices 212A/B. As illustrated in FIG. 8, the first 214A and second 214B members of the pairs of extendable/retractable lattices are identical. Each member of the pair comprises two identical equal lateral geometric diamonds 215A and 215B and 215C and 215D (parallelograms with opposite sides parallel. But the body does not form a square with right angles at every corner; in the following disclosure, all sides are of equal length).

The first diamond 214A of the first lattice 214 comprises two identical connector arms 216A and 216B. The arms 216A and 216B terminates in a first or second lower gear segment 217A and 217B, respectively, that functionally engage each other and are physically and functionally connect the corresponding lower anchor connector bracket 209A by an axle that allows the gears to rotate through an opposing 180 degree arcs.

Arm 216 extends from the center point of the first lower gear 217A to pivot point 218A and arm 216B extends from center pivot point of gear 217B to pivot point 218B. Arm 220 shares a pivot point 219 at the midpoint of arm 220A and 220B. Arm 220B extends from pivot point 218B to pivot point 221A (Note the shared pivot point with arm 220A.)

Arm 222A extends from pivot point 221A to the center point of the first upper gear 223A, and arm 222B extends from pivot point 221B to the center point of the second upper gear 223B. This completes the connections and arms forming the members of the first pair 214 diamonds 214A and 214B, respectively.

The members of the second pair 215 of diamonds 215A and 215B are connected in the same pattern; the two pairs of diamonds 214 and 215 are identical. Starting at the lower, anchor connector bracket 209B, arm 225A is connected to center point of the second lower gear 230A and to pivot point 224A, and arm 225B is connected to center point of gear 230B and to pivot point 224B. Arm 226 extends from pivot point 224A to pivot point 228B with shared pivot point 225 in the center of arm 226 and 227. Arm 227 extends from pivot point 224B to pivot point 228A, via shared pivot point 225. Arm 230A extends from pivot point 228A to center point of gear 231A, and arm 230B extends from pivot point 228B to center point of the second upper gear 231B.

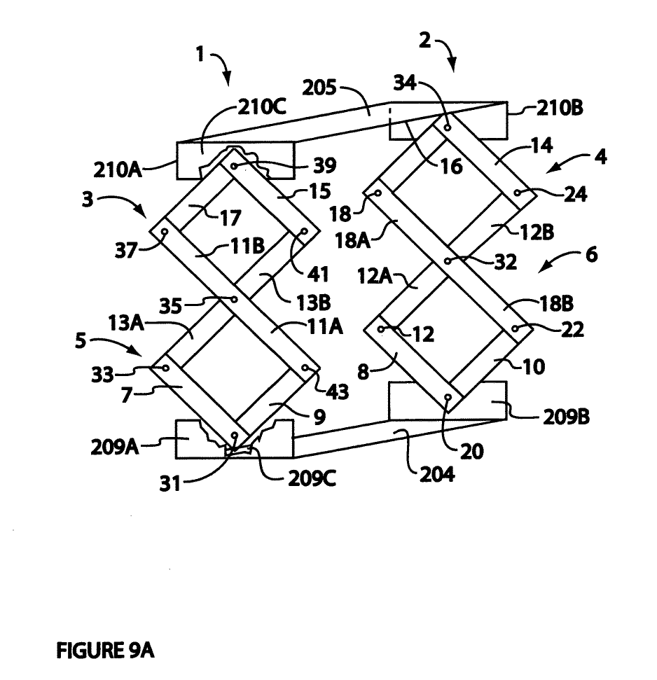

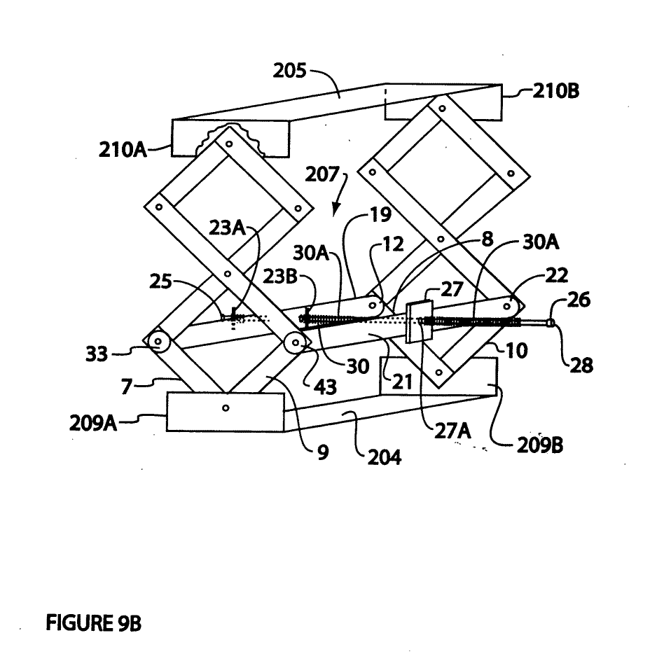

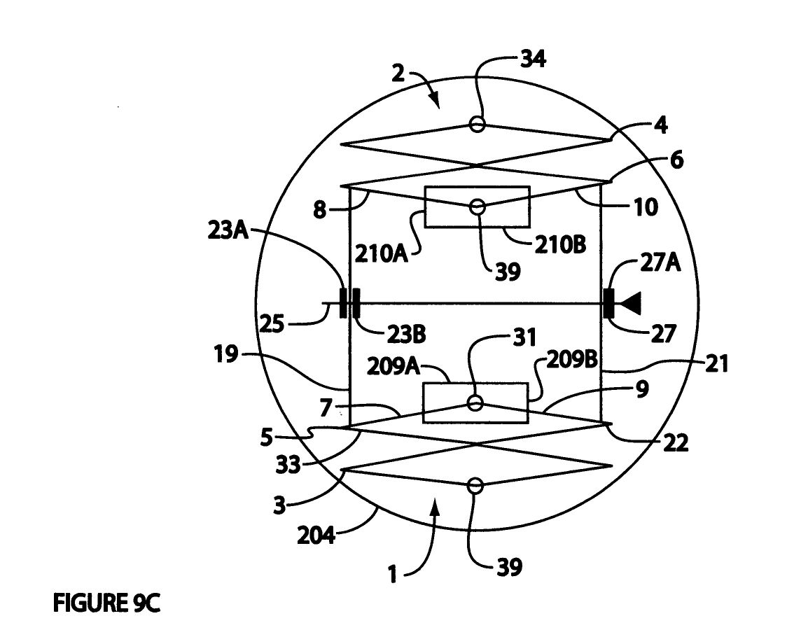

FIG. 9A illustrates a modification of the connection of the members of the pair of lattices 214 and 215 to the corresponding lower and upper anchors connector brackets 209A/B and 210A/B. In FIG. 8, arms 216A and 216B and 222A and 222B and arms 225A and 225B and 230A and 230B are connected to (or effectively part of) separate gears that are functionally engaged and are in pairs that are physically part of the corresponding arm. In FIG. 9A, the pairs of arms 216A/216B, 230A/230B, 225A/B, and 230A/B are physically connected and are connected to the corresponding lower and upper lift anchor connectors 209A and 209B and 210A and 210B, respectively. In terms of function, this represents no change in the scope or purpose of the invention or modification in the basic structure or function of the lift mechanism. The alternative may affect manufacturing and/or costs, but not the application or function of the device 201 as used in practice by the surgeon.

Areas designated 209C or 210C indicate cut away illustrations to show details in 204 or 205.

Each member, 1 and 2, of the pair of lattices comprises four short arms and two long arms as shown in FIGS. 9A and 9B. The long arms, taken from connection point to connection point, from the first end to the second of the arm, are twice as long as the short arms. The short arms in each lattice 7 and 9 and 15 and 17 for diamonds 5 and 3 in lattice 1, and 8 and 10 and 14 and 16 for lattice 2, diamonds 6 and 4.

The first end of the members of each pair of short arms are pivotally connected to an anchor connector bracket. For example, the first ends of short arms are connected, and by the same device (bolt, rivet, or very short shaft or comparable means, as one skilled in the art understands), the arms are connected to anchor connector bracket 209A at point 31.

The first lattice 1 further comprises a pair of long arms. Each long arm is divided into equal halves, with a pivotal center point, and both halves of each long arm are identified as to the long arm and first of second half as follows.

For lattice 1, the long arms 11A/11B and 13A/13B. Note that each half of a long arm has a single, free end; the second end effectively is the common, central pivot point. Thus, The first end of 13A is pivotally connected to the second end of short arm 7 at point 33 and the first end of 11A/B, first end 11A is pivotally connected to the short arm 9 at point 43. The two long arms 11A/B and 13A/B extend diagonally upward and cross at midpoint 35 at which point they are pivotally connected. The midpoint separates the two segments of each long arm 11A/11B and 13A/13B. The first (free) end of 11B is pivotally connected to the second end of short arm 17 at point 37 and the first (free) end of 13B is pivotally connected to the second end of short arm 14 at pivot point 24, and the first ends of short arms 14 and 16 are pivotally connected at point 39 at which point they are also pivotally connected to anchor connector bracket 209B at point 39.

FIG. 9A also shows the position and general structure of the lift mechanism 207. As illustrated, the mechanism comprises three main parts: a pair of drive connector rods 242A and 242B and a gear drive rod 30. The first drive connector rod 242A is positioned between pivot point 218B and pivot point 224B, and the second drive connector rod 242B is positioned between pivot points 218B and 242B.

At each of the sets of nine pivot points, the connection is made with connector axle/bolt. For connecting the gear with the corresponding bracket: 217A/B with 209A; 223A/B with 209B; 223A/B with 210A; and, 229A/B with 210B, the connector bolt (or comparable device) connects the gear end of the arm at the center point of the gear to the corresponding lower or upper lift anchor connector bracket 209A/B and 210A/B, respectively. The first (distal) end segment of the connector bolt is threaded to engage a nut to secure the bolt/axle in position; the second (proximal) end segment is smooth and functions as an axle around which the arms rotate, or travel in an arc.

FIG. 9A and 9B illustrate a device in which the gears of FIG. 8 are not required, and this model provides the basis following operational model and discussion of the device. As one skilled in the art recognizes, inclusion or exclusion of the gears does not change or intent of the invention. With gears, there are two pairs of gear pivot points for each lattice, 1 and 2, a pair for each of the four diamonds (two pairs) described by the arms with the first pair designated 3 and 5 and the second pair designated 4 and 6 for lattices 1 and 2, respectively. Without gears, the two arms are joined at a single pivot point 33 and 43 and 12 and 22, respectively for 1 and 2 lattices and (for diamonds 3 and 5, lattice 1) and (for diamonds 4 and 6, lattice 2), respectively. Index number identification for the lower and upper base plates, 204 and 205 respectfully, and for the pairs of lower and upper anchor connector brackets 209A and 209B and 210A and 210B, respectively remain the same in FIGS. 9A, B, and C as in FIGS. 6 and 7.

From the above, one skilled in the art understands that the number of diamonds can be increased by adding at least one pair of long arms to the structure. Increasing the number of long arms increases the maximum length (height) to which the lattice can be extended. Increasing the number of diamonds does not alter the design, in that the top and bottom of each lattice are attached to the corresponding anchor connector brackets by a pair of short arms. This modification does not alter or extend the scope or intent of the invention or of the appended claims and is anticipated and claimed without further illustrations or discussion.

The device is anchored between the femur and tibia, the upper 210A/B and lower 209A/B anchor connector brackets, respectively, secured to the lower base plate (tibia) 204 and upper base plate (femur) 205. The first 19 and second 21 connector elements effectively connect the second (index number/line 5) and fourth (index number/line 6) diamonds at pivot points 43 and 22 and at pivot points 33 and 12 in the same diamonds (index numbers 5 and 6). For reference purposes, lattices 1 and 2 are mutually complimentary; diamond 1 (index number 3 in lattice 1) and diamond 3 (index number 4 in lattice 2) are complimentary as are diamond 2 (index number 5, lattice 1) and diamond 4 (index number 6, lattice 2). In addition, within a diamond, e.g. diamond 2 (index number 5) pivot points 33 and 43 are alternative pivot points as are pivot points 12 and 22 in diamond 4 (index number 6). Directly opposite pivot points in complimentary diamonds are complimentary, and a pivot point in in one diamond may have only complimentary pivot point in any given complimentary diamond. The addition of a pair of long arms in each lattice results in the formation of a single additional diamond in each lattice and a correlated increase in maximum extension height of the adjustable, tibial trial device.

The drive rod 30 comprises a stainless rod (or a rod of comparable material) with a first end 25 and a second end 26. The drive rod unit 30 is threaded 30A from the first end 25 for about 80 percent of the length of the drive rod 30 and smooth for the remaining length of the drive rod 30 to the second end 26. The second end 26 of the drive rod unit 30 is adapted to engage an instrument (or tool) 28 to manually rotate the drive rod 30 as it is engaged with the threaded rod chase 27A of the travel block 27. The travel block 27 is a part of the second lift connector 21 that comprises a threaded chase 27A at its longitudinal mid-point through which the drive rod 30 traverses to extend (or contact) vertically the lattices- increase or decrease the height of the lattices in response to rotation of the drive rod through the drive rod travel block 27. In addition, members of a pair of travel lock pins or comparable devices 23A and 23B are positioned through the drive rod 30 on opposite sides of the first lift connector 19 and a the smooth segment of the drive rod unit to prevent unwanted lateral movement of the threaded drive rod 30 as the threaded drive rod 30 is rotated. The drive rod 30 traverses the diameter of both the first and second lift connectors 19 and 21 by means of a smooth wall chase that is functionally aligned with threaded chase 27A.

FIG. 9A illustrates the adjustable tibial trial device in a partially deployed or open (extended) configuration. Perspective of the illustration may suggest that the pair of lower spacing anchors 209A/B and upper spacing anchors 210A/B are unequal in height. They are equal in height. Lengths to pivot point 31 to 39 and 20 to 34 are equal, and FIG. 9B illustrates the device in a nearly completely retracted or closed configuration. The numbering of the arms and pivot points follows that of FIG. 9A; the upper base plate 205 and upper lift anchor 208 are not shown in FIG. 9B. Note, to achieve a 3-dimensional effect in FIG. 9A, lattice 1 is on the left side and appears to be slightly forward of lattice 2; whereas, in FIG. 9B, lattice 1 is at the bottom of the figure and lattice 2 is at the top of the illustration, thereby providing a simple, 2-dimensional perspective.

One skilled in the art recognizes that the maximum extension of the lattices is increased by adding pairs of long arms to each lattice. Increasing this does not change the scope or intent of the invention as expressed in the appended. claims and such modifications are anticipated by this disclosure and claims.

Considering FIG. 9B, with both lattices 1 and 2 nearly fully retracted or closed, with short arms 7 and 9 connected at pivot point 31, connector bracket 209A, and short arms 8 and 10 connected at pivot point 20, connector bracket 209B The downward compression moves pivot points 33 and 22 down resulting in the distortion of diamonds 3 and 5 in lattice 1 and diamonds 4 and 6 in lattice 2. All pivot points move downward contributing to the flattening (distortion) of the diamonds as the lattices are retracted.

The first and second connector units 19 and 21 respectively connect the two lattices by pivotal connections at opposing pivot points 43 and 22 (second connector unit 21) and pivot points 33 and 12 (first connector unit) on the lattices.

As the lattices retract and the diamonds distort to horizontally long, narrow structures, the pairs of short arms 7/9 and 8/10 ultimately rest on the surface of the lower base plate 204. The pairs of long arms 11A/B and 13A/B (lattice 1) and 12A/B 18A/B (lattice 2) are aligned generally parallel to each other along the surface of the lower base plate 204, and the second pair of short arms 17/18 and 16/14 are aligned the surface of the base plate 204.

The minimum thickness of the device equals the width of the arms (assumed, but not required to be 3 mm) plus the thickness of the lower base plate (minimum 2 mm) plus the thickness of the upper base plate (2 mm) or 13 mm, approximately 0.5 in.

Functionally, the drive 30 is rotated so that so that the drive rod 30 apparently extends forward through the travel block 47. In fact, with the travel block 27 anchored to the second lift connector 21 and the ends of the second lift connector 21 connected pivotally to pivot points 35 and 38, actually rotation of the drive rod 30 moves the travel block 27 and attached second lift connector 21 backwards towards the second lattice. This horizontal movement is converted to vertical movement at pivot point 35 as a result of the total arrangement and anchoring of the arms in both latices.

Because arms 13A and 13B are a single, continuous unit and pivot around pivot point 35 in the first lattice, as pivot point 35 moves, as viewed in FIG. 9, from left to right, pivot point 41 moves from right to left; effectively, the angle at pivot point 41 becomes increasingly vertical as arms 13B and 15 move inward, with arms moving upward, elevating pivot point 39, and effectively increasing height.

As arms 7 and 13A move inward, arms 9 and 11A respond as described above for arms 7 and 13. Arms 11A and 11B, just as arms 13A and 13B, are a single, continuous unit and pivot around pivot point 33. The extension of lattice 1 is uniform; pulling arms 11B and 17 increases height of pivot point 39, the same as increasing the distance between the lower and upper base plates 204 and 205. Movement on both sides of lattice 1 is concurrent and interdependent between the sides of the lattice.

Because the first and second lift connectors 19 and 21, respectively, are functionally secured to a corresponding points in the first lattice, points 43 and 22, and the second lattice 33 and 12, the various combinations of arms in the second lattice react simultaneously in the same manner and magnitude as in the arms of the first lattice; thus, the lift is uniform. Increasing lift (height) can be stopped merely by stopping the manual rotation of the drive rod unit. The lattice can be lowered by reversing the direction of rotation of the drive shaft unit. A variety of devices from a snap ring to a threaded nut may be positioned on the first end 25 of the drive rod 30 to prevent inadvertent rotation of the drive shaft and lowering the device before necessary measurements are completed.

The adjustable, tibial trial device provides a mechanism to continuously increase (or decrease) the gap or space between the femur and tibia caused by preparation of these bones, including removal of cartilage for implant of a prosthetic knee joint and a space (gap) which ultimately must be properly filled by an appropriate spacer that is part of the implant.

Rather than depending on trial and error, frequently repeated fittings of trial spacers, the adjustable, tibial trial allows the surgeon to increase the spacing, and by examination determine if that spacing is optimal and to reduce or further increase the spacing until examination confirms an appropriate fit has been achieved.

FIG. 9C illustrates the adjustable, tibial trial in a nearly fully closed or retracted configuration.

The numbering of the arms and pivot points follows that of FIG. 9A, and the upper base plate 205 and upper spacing anchor 208 are not shown. Note, to achieve a 3-dimensional effect in FIG. 9A, lattice 1 is on the left and appears slightly forward of lattice 2; whereas, in FIG. 9B, lattice 1 is at the bottom and lattice 2 is at the top of the illustration, as the device appears in a simple, 2-dimensional perspective.

FIG. 9C shows the device with both lattices 1 and 2 nearly fully collapsed, with arms 7 and 8 connected at pivot point 39 at anchor connector brackets 209A and 209B, and as a result, diamonds 3 and 5 in lattice 1 and of diamonds 4 and 6 in lattice 2 follows and the arms are arrayed virtually parallel on the base plate. The first and second connector units 19 and 21 respectively are connected to opposing pivotal points in diamonds 5 and 6. When collapsed, the bottom most, short arms (7/9 and 8/10, FIG. 9A) are on the surface of the lower base platform 204. The pairs of long arms in each lattice 11A/B and 13A/B in lattice 1 and 12A/B and 18A/B in lattice 2 (FIG. 1A) are stacked as pairs, and other short arms are stacked parallel to them; thus, the minimum thickness of the adjustable, tibial trial is less than twice the thickness of any arms plus the thickness of the lower and upper plates. By way of example, not limitation, this thickness may range from 10 to20 mm, about 0.33 in to 0.80 in.

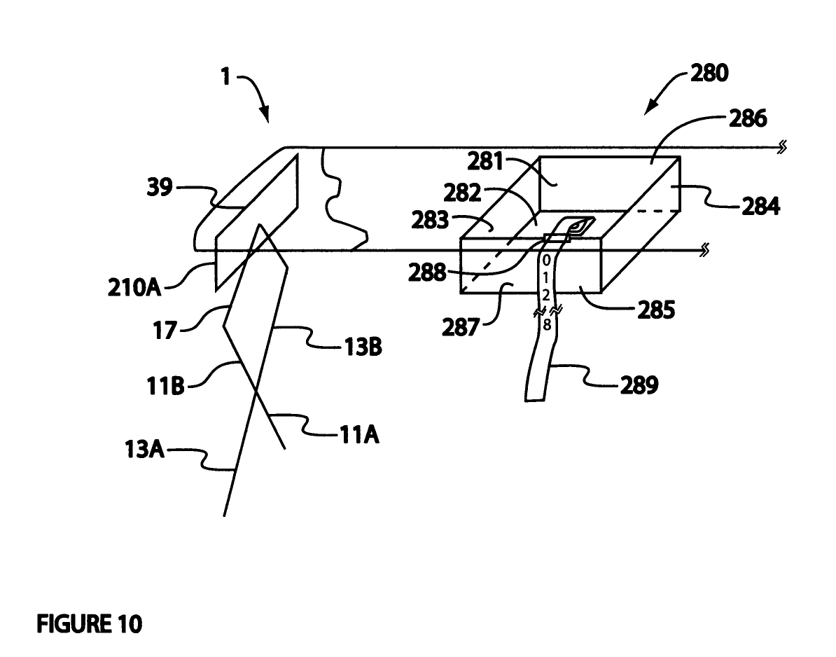

One of a potential variety of elements to measure the distance between the surface of the femur and the surface of the tibia (the required thickness of the spacer to ensure appropriate tension on support ligaments as determined by the surgeon is illustrated in FIG. 9 and FIG. 10.

FIG. 10 illustrates one of a variety of possible measuring tape housings or enclosures 280 comprising, as illustrated, a top 281, a bottom 282, a first side wall 283 a second side wall 284, a front wall 285, and a back wall 286 is secured to the inward facing surface of the upper base plate between the members of the upper lift anchor connectors; in FIG. 10, only the first member 10A of a pair is shown for simplicity, without affecting the information provided by the Figure. The six-sided measuring tape housing 280 is assembled with the top and bottom edges of the front wall, the back wall, and the two side walls are secured to are secured to the edges of the top 281 and bottom 282 and all adjacent edges and corners, except the front wall may be removably connected to the front edges of the top and bottom and to adjacent edges of the two side walls. The measuring tape housing 280 further comprises a measuring tape, the proximal end of which is secured to the inner surface of the bottom 282 and the tape is folded serpentine fashion such that it can be readily pulled (extended) full length from a slot on the lower edge of the front wall 285. The reference, “0” point is at the edge of the opening, at the edge of the femoral plate, or at the edge of the femur and the tape extends downward to a comparable point for the tibia, the surface of the tibial plate or of the tibia. Measurement for the thickness of the spacer are adjusted simply by adjusting the distance of the “0” point at the measuring tape housing to the desired top of the spacer and of the bottom point, both of which distances are fixed. The tape is marked in one or two mm increments, and spacers are most commonly supplied in 2 mm increments.

Major providers of orthopedic implants, such as Smith & Nephew (noted without endorsement) provide a wide array of sizes based on two measurements the A/P anterior to posterior distance and the M/L medial to lateral length. Dimension vary from femur to tibia with eight sizes available for each. The femoral dimensions range for the smallest (size 1) A/P 47.0 mm and M/L55.0 mm; size 5 A/P 62.0 and M/L 70.0 mm; and the largest (size 8) A/P 75.0 mm and m/L 80.0 mm. Overall, the comparable tibial values are greater: smallest (size 1) A/P 42.0 mm and M/L 60.0 mm; size 5 A/P A/P 52 mm and M/L 74 MM; and the largest (size 8) A/P 59.0 mm and M/L 85 mm. Thus, the surgeon is faced with a wide array of combinations; the aspect ratio of the tibia A/P:M/L is given added weight in decision making.

What I claim is:

1. An adjustable tibial trial device comprising five components: (1) a lower base plate, (2) an upper base plate, (3) a pair of lower anchor connector brackets, (4), a pair of upper anchor connector brackets, and a spacing mechanism; wherein, said lower base plate is removably positioned on the proximal tibia and said upper base plate is removably positioned on the distil femur; and,
wherein, the members of said pair of lower anchor connector brackets are aligned mutually parallel on said lower base plate, and members of said pair of upper anchor connector brackets are aligned mutually parallel on said upper base plate; and wherein said member of said first pair of lower anchor connector brackets and of said first pair of upper anchor connector brackets are functionally vertically aligned, and the members of said pair second pair of said lower anchor connector brackets and the second pair of the upper anchor connector brackets are functionally vertically aligned and both members of both of anchor connector brackets to a corresponding base plate; and,
wherein said spacing mechanism comprises a pair of vertically extendable/retractable lattices, and wherein each member of said pair of lattices comprises two pairs of short arms and at least one pair of long arms, wherein, for each of said lattices, the first ends of the member of the first pair of short arms are pivotally connected to the first lower anchor connector bracket, and the first ends of the second pair of short arms are pivotally connected to the first upper connector bracket; and wherein the first end of the first member of said at least one pair of long arms is connected to the second end of said first pair of short arms, and the second of said first pair of short arms and the first end of the second member of said at least one pair of long arms is joined to the second end of said second member of said first pair of short arms and wherein said first and second members of said pair of long arms are pivotally joined at their mutual midpoint, and wherein the second end of said first member of said pair of long arms is pivotally connected to the second end of said second pair of short arms; and wherein, for said second lattice, two pairs of short arms and at least one pair of long arms functionally are connected to said second lower and second upper anchor connector brackets and connected by said the members of said at least one pair of long arms to form said second lattice;
one member of a pair of lift connector elements connects a point in a diamond formed by the arms in said first lattice to a complimentary point in a diamond formed by the arms of said second lattice, and the second member of said pair of lift connectors elements secures a second point in said diamond in said first lattice with at complimentary point in said complimentary diamond in said second lattice; the first end of the first member of said pair of lift connector elements is secured to a point in a diamond of said first lattice, and the second end of said first lift connector element is is secured to a complimentary point in the complimentary diamond of said second lattice; the second member of said pair of lift connector elements is similarly connected to the same complimentary diamonds in each lattice, and the member of the pair of lift connector elements are horizontally parallel when positioned in complimentary diamonds and connected to complimentary pivot points;
said spacing mechanism further comprises a threaded drive rod and a travel block, and, wherein, said travel block is secured to the midpoint of one of said pair of lift connector elements and wherein threaded travel chase traverses said travel block in a horizontal line as a smooth walled (un-threaded) chase through both of said lift connector elements, and wherein said threaded travel chase is adapted functionally adapted to engage said threaded travel chase, and said threaded drive rod is adapted to extend through said smooth walled chase and the second end of said threaded drive shaft is adapted to engage a tool wherein said tool manually rotates said drive shaft and wherein a section of the first end of said drive shaft is not threaded and does not mechanically engage the smooth walled chase when said tool is rotated.

2. The adjustable, tibial trial device of claim 1, wherein, for each member a pair of lattices the first end of the first pair of short arms to the first member of a first pair of gears and wherein said first member of said first pair of gears is functionally, pivotally attached to said first, lower anchor connector bracket, and, wherein the first end of the second member of said first pair of short arms is functionally attached to the second member of said first pair of gears, and, wherein, second member of said first pair of gears is pivotally attached to said second lower connector bracket, and wherein, said first member and said second member of said first pair of gears are spaced to be functionally engaged; and, wherein the first end of the first member of the second pair of short arms is functionally attached to the first member of a second pair of gears, and, wherein, said first member of said second pair of gears is functionally and pivotally attached to said first, upper anchor connector bracket, and the first end of the second member of said second pair of short arms is is functionally attached to to the second member of said second pair of gears and wherein said second member of said second pair of gears is functionally, pivotally attached to said second upper anchor connector bracket; and where said first member of said second pair of gears and said second member of said second pair of gears are space to be functionally engaged.

3. The adjustable tibial trial device of claim 1, wherein, each lattice must have and additional two pairs of long arms.

4. The adjustable tibial trial device of claim 1, wherein said device further comprises a measurement tape housing.
